(19) United States
(12) Patent Application Publication
(10) Pub. No.: US 2025/0306398 A1
(43) Pub. Date: Oct. 2, 2025

(54) METHOD FOR ANCHORING AMNIOTIC MEMBRANE TO A CONTACT LENS CARRIER

(71) Applicant: Christopher Adams, somerville, MA (US)

(72) Inventor: Christopher Adams, somerville, MA (US)

(21) Appl. No.: 19/090,714

(22) Filed: Mar. 26, 2025

Related U.S. Application Data
(60) Provisional application No. 63/571,658, filed on Mar. 29, 2024.

(51) Int. Cl. G02C 7/04; A61B 50/31; A61F 9/00

(52) U.S. Cl. CPC G02C 7/04; A61B 50/31; A61F 9/0017; A61B 2050/311

(57) ABSTRACT

This document describes various methods for attaching biological tissues, such as amniotic membranes, to contact lenses. The techniques include using anchor points on the lens surface, employing biological or synthetic glues, and utilizing a piggybacking method. The materials involved in these processes can include silicon hydrogel, collagen, or other hydrogel materials, which may be loaded with growth factors or pharmaceutical agents. Additionally, the piggybacking configuration is designed to degrade or be bioabsorbed over a period of 1-10 days on the ocular surface.

### TECHNICAL FIELD

The present invention relates to a method for anchoring an amniotic membrane to a contact lens carrier, and more particularly, to a method for anchoring an amniotic membrane of approximately 8-14 mm in diameter with a thickness of 100-500 microns to a contact lens or other carrier with or without any fixture solvents or formulations.

### BACKGROUND

In the field of ophthalmology, the use of amniotic membranes has become increasingly prevalent for the treatment of various ocular surface diseases. These membranes, derived from the placenta, are known for their anti-inflammatory and healing properties, making them an ideal choice for promoting recovery in damaged corneal tissues. Traditionally, the application of amniotic membranes in eye care involves suturing or using adhesion materials to attach the membrane to the eye, which can lead to complications such as infection, discomfort, and delayed healing. As a result, there is a growing need for more efficient and less invasive methods to secure amniotic membranes in place.

Contact lenses have long been used as therapeutic devices in ophthalmology, providing a protective barrier and delivering medication directly to the eye. The integration of amniotic membranes with contact lenses presents a promising solution to enhance the therapeutic benefits while minimizing the complications associated with traditional attachment methods. However, existing techniques for combining these two components often involve complex processes or additional materials that can compromise the effectiveness and comfort of the treatment. Therefore, there is a significant demand for innovative approaches that simplify the integration of amniotic membranes with contact lenses, ensuring stability and efficacy without the drawbacks of conventional methods.

### SUMMARY

A method may involve attaching amniotic tissue to a contact lens by using anchor points on the lens surface. These anchor points can consist of 1-100 discrete structures designed to capture and hold the amniotic tissue in place.

The method may include molding or fabricating the lens and anchor points in a single or multi-step process.

The lens and anchor material can be composed of silicon hydrogel, collagen, or hydrogel material commonly used in the contact lens material field.

The biological tissue used in the method may be an amniotic membrane, chorion material, or some examples of functional tissue.

The hydrogel material can be applied as a disc loaded with growth factors or pharmaceutical medicaments.

A method may involve attaching amnion or biological tissue to a contact lens surface using biological or synthetic glues, such as fibrin, sucrose, or cyanoacrylate.

A method may involve attaching amniotic or biological tissue to a contact lens surface using a piggybacking method.

The piggybacking lens material can be silicon hydrogel, collagen, or hydrogel material and may be functionally permeable to small molecules or proteins.

The piggybacking configuration may degrade or be bio-absorbed over a 1-10-day period on the ocular surface.

The amniotic membrane may be manufactured with contact lenses in the method.

The amniotic material can be either molded or placed in the contact lens material prior to polymerization.

The amniotic material may be either solid, micronized, or liquid.

### BRIEF DESCRIPTION OF THE DRAWINGS

FIG. 1 shows a contact lens with peripheral slots to secure an amniotic membrane.

FIG. 2 shows a contact lens with anchor points for securing an amniotic membrane, stored in a container.

FIG. 3 shows a piggyback configuration with an upper and lower contact lens enclosing amnion material.

### DETAILED DESCRIPTION

This disclosure provides a method for anchoring an amniotic membrane to a contact lens carrier, which may involve using anchor points, slots, or ridges to secure the amniotic tissue. The anchor points can be molded or fabricated in a single or multi-step process, and the lens and anchor material may be composed of silicon hydrogel, collagen, or other hydrogel materials. The amniotic membrane, which can be an amniotic membrane, chorion material, or other functional tissue, may be applied to the contact lens without any fixture solvents or formulations. The hydrogel material can be applied as a disc loaded with growth factors or pharmaceutical medicaments to promote healing and biological effects. Additionally, the method may involve attaching the amniotic or biological tissue to the contact lens surface using biological or synthetic glues, such as fibrin, sucrose, or cyanoacrylate. Alternatively, a piggybacking method may be used, where the piggybacking lens material is functionally permeable to small molecules or proteins and can degrade or be bioabsorbed over a 1-10-day period on the ocular surface. The amniotic membrane may be manufactured with contact lenses, and the amniotic material can be either molded or placed in the contact lens material prior to polymerization, allowing for flexibility in the material state as solid, micronized, or liquid.

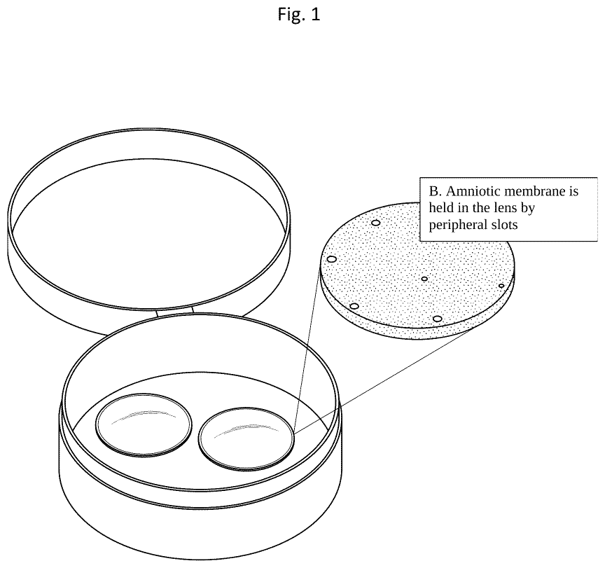

Referring now to FIG. 1, the illustration depicts a contact lens designed to secure an amniotic membrane using peripheral slots, as part of a method for anchoring amniotic tissue to a contact lens. The contact lens is shown within a case, highlighting the approach of using slots as anchor points to hold the amniotic membrane in place. This design addresses the need for a secure attachment method without relying on additional adhesives.

The contact lens is depicted with distinct slots around its periphery, which serve as anchor points for the amniotic membrane. These slots are strategically positioned to ensure that the membrane remains securely attached to the lens surface. The design may involve 1-100 discrete structures to capture and hold the amniotic tissue effectively. The slots can vary in size and shape, providing flexibility in accommodating different tissue dimensions and ensuring a snug fit.

The structural relationship between the contact lens and the amniotic membrane is important for maintaining the membrane's position on the ocular surface. The peripheral slots are designed to interact with the membrane, providing a mechanical grip that prevents displacement. This method eliminates the need for adhesives, reducing the risk of contamination and irritation, which are common issues with traditional attachment methods.

In terms of functionality, the slots not only secure the amniotic membrane but also allow for easy application and removal of the lens. This design can be fabricated using materials such as silicon hydrogel, collagen, or other hydrogels common in the contact lens field. The integration of the amniotic membrane with the contact lens through these slots enhances the therapeutic potential of the lens, allowing for the delivery of biological growth factors and facilitating healing on the ocular surface.

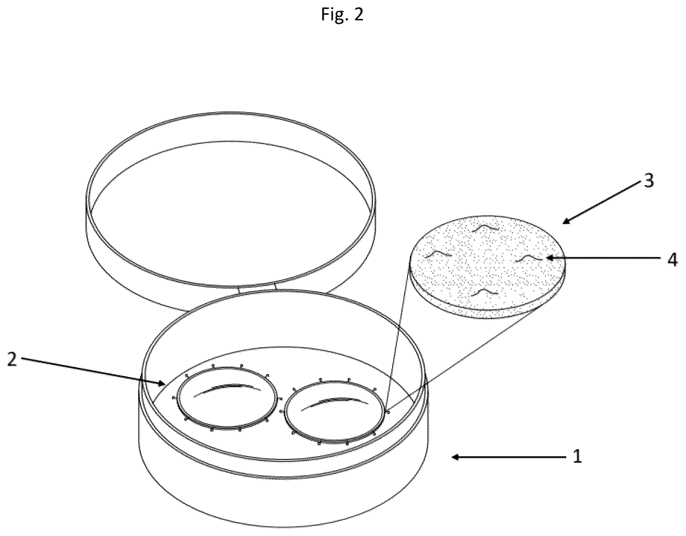

Referring now to FIG. 2, the technical drawing illustrates a contact lens designed to secure an amniotic membrane using anchor points. The container (1) holds the contact lens (2), which is equipped with multiple anchor points (4) around its periphery. These anchor points are discrete structures intended to capture and hold the amniotic membrane (3) in place. The anchor points may vary in number from 1 to 100, providing a mechanism for securing the biological tissue to the lens surface. This configuration ensures that the amniotic membrane remains securely attached, reducing the risk of displacement when applied to the ocular surface.

The contact lens (2) is designed to integrate with the amniotic membrane (3) through these strategically placed anchor points (4). These points can be molded or fabricated in a single or multi-step process. The lens and anchor points may be composed of materials such as silicon hydrogel, collagen, or other hydrogels common in the contact lens field. The anchor points are important for maintaining the position of the amniotic membrane, which can be composed of various biological tissues, including amniotic membrane, chorion material, or other functional tissues.

The structural relationship between the contact lens and the amniotic membrane is enhanced by the anchor points, which provide a secure attachment without the need for additional adhesives or binding agents. This method addresses the drawbacks of previous techniques that relied on binding agents, which could cause irritation or interfere with healing. The anchor points ensure that the amniotic membrane is held firmly in place, allowing for effective treatment of ocular surface diseases. The container (1) serves as a protective case for the lens, ensuring that the anchor points and the amniotic membrane remain intact and ready for application. This design offers a practical solution for integrating biological tissues with contact lenses, enhancing the therapeutic potential of the device.

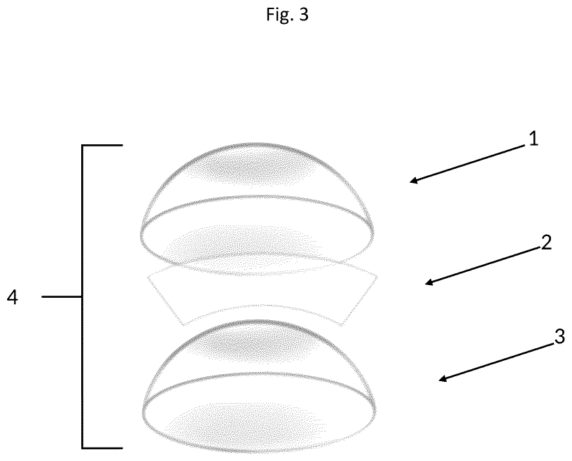

Referring now to FIG. 3, the illustration depicts a piggyback configuration of an amnion lens complex. This configuration involves an upper contact lens (1) and a lower contact lens (3) that enclose the amnion material (2), forming the amnion lens complex (4). The upper contact lens (1) and lower contact lens (3) are designed to sandwich the amnion material (2) effectively, allowing for the diffusion of biological molecules and providing a cushioning effect. This setup is beneficial for reducing discomfort and enhancing the therapeutic application of the amnion material on the ocular surface.

The upper contact lens (1) and lower contact lens (3) may be composed of materials such as silicon hydrogel, collagen, or other hydrogels that are permeable to small molecules or proteins. This permeability is important for the diffusion of biological molecules, such as cytokines and growth factors, from the amnion material (2) to the ocular surface. The amnion material (2) itself is strategically placed between the two lenses to ensure it remains in position and can effectively deliver its therapeutic benefits. The amnion lens complex (4) thus formed is designed to degrade or be bioabsorbed over a period of 1-10 days, providing a controlled release of therapeutic agents.

The structural relationship between the components is integral to the functionality of the device. The upper and lower lenses (1 and 3) are positioned to create a secure enclosure for the amnion material (2), ensuring it remains in place during use. This configuration not only facilitates the diffusion of therapeutic agents but also provides a cushioning effect, reducing any potential discomfort from the contact lens. The interaction between these components is designed to optimize the therapeutic efficacy of the amnion material while maintaining comfort for the user. This approach addresses the need for improved methods of anchoring amniotic membranes to contact lenses.

Example 4 illustrates a method for attaching amniotic tissue to a contact lens using anchor points on the lens surface, according to an embodiment. At step 1, the attachment of amniotic tissue to a contact lens may be facilitated by utilizing anchor points on the lens surface. These anchor points can be designed to capture and hold the amniotic tissue in place, potentially ranging from 1 to 100 discrete structures. The lens and anchor points may be molded or fabricated in a single or multi-step process, allowing for a secure attachment of the tissue to the lens. The lens and anchor material may be composed of silicon hydrogel, collagen, or hydrogel material, which are common in the contact lens material field. The biological tissue used may be an amniotic membrane, chorion material, or other functional tissue. The hydrogel material may be applied as a disc loaded with growth factors or pharmaceutical medicaments, enhancing the functionality of the lens. The use of anchor points and ridges may hold the amniotic membrane securely, while the slots, attachment points, or anchor points may ensure the tissue is secured effectively. The amniotic membrane may be anchored to a contact lens carrier, providing a comprehensive device that may facilitate the incorporation of amniotic properties into the lens. The use of growth factors and pharmaceutical medicaments may promote healing and biological effects, potentially combining with the amniotic membrane material to enhance the overall functionality of the lens.

Example 5 illustrates a method in step for attaching amnion or biological tissue to a contact lens surface using biological or synthetic glues, according to an embodiment. At step 1, the attachment of amnion or biological tissue to the contact lens surface may be facilitated through the application of biological or synthetic glues, such as fibrin, sucrose, or cyanoacrylate. The process may involve the use of gelatin hydrogels or sugar-based glues to ensure the secure attachment of the amniotic tissue to the lens. The biological or synthetic glues may serve as adhesives that provide a stable interface between the amniotic tissue and the contact lens surface. The use of fibrin glue, for instance, may offer a biologically derived tissue sealant that can control bleeding and provide a secure attachment. Cyanoacrylate or gelatin hydrogels may also be employed as bio adhesives to attach the biological tissue to the lens. The sugar-based glue, such as a sucrose mixture, may be applied at specific points on the lens surface, allowing the amnion tissue to be placed over it. As the sucrose glue dries, it may form a tissue-lens-glue complex, ensuring the stability of the attachment. The lens device complex may be stored in saline solution, frozen, or dehydrated to maintain its stability over an extended period. Once placed on the eye, the glue and amnion may begin to degrade due to the ocular environment, including increased temperature and enzymatic degradation. This method may provide a secure and efficient means of attaching amniotic or biological tissue to a contact lens surface, potentially enhancing the therapeutic benefits of the lens.

Example 6 illustrates a method for attaching an amniotic or biological tissue to a contact lens surface using a piggybacking method. In this method, the piggybacking configuration may involve the use of a soft contact lens placed beneath a rigid gas permeable (GP) lens to reduce discomfort and secure the amnion. The piggybacking lens material may be composed of silicon hydrogel, collagen, or hydrogel material, which is functionally permeable to small molecules or proteins. This permeability may allow for the diffusion of biological molecules, enhancing the functionality of the lens. The piggybacking configuration may degrade or be bioabsorbed over a period of 1 to 10 days on the ocular surface, utilizing bioerodible materials to facilitate this process. The amniotic membrane may be manufactured with contact lenses, allowing for the integration of amniotic properties into the lens. The amniotic material may be either molded or placed in the contact lens material prior to polymerization, providing flexibility in the material state as it can be solid, micronized, or liquid. This incorporation of the amniotic composition into the polymeric material may create a lens with embedded amniotic properties, potentially enhancing the therapeutic benefits of the lens. The method may offer an alternative attachment method that reduces discomfort while securing the amnion effectively.

What is claimed is:

1. A method of attaching amniotic tissue to a contact lens using anchor points on the lens surface. Such anchor points can be from 1-100 discrete structures designed to capture and hold the amniotic tissue in place.

2. The method according to claim 1 where the lens and anchor points are molded or fabricated in a single or multi-step process.

3. The method according to claim 1 where the lens and anchor material are composed of silicon hydrogel, collagen or hydrogel material common in the contact lens material field.

4. The method according to claim 1 where the biological tissue is an amniotic membrane, chorion material or other functional tissue.

5. The method according to claim 1 where the hydrogel material can be applied as a disc loaded with growth factors or pharmaceutical medicaments.

6. A method of attaching amnion or biological tissue to a contact lens surface using biological or synthetic glues, such as fibrin, sucrose or cyanoacrylate.

7. A method of attaching an amniotic or biological tissue to a contact lens surface using a piggybacking method.

8. A method according to claim 7 where the piggybacking lens material is silicon hydrogel, collagen or hydrogel material and is functionally permeable to small molecules or proteins.

9. The method according to claim 7 where the piggybacking configuration degrades or is bio absorbed over a 1-10-day period on the ocular surface.

10. The method according to claim 7 where the amniotic membrane is manufactured with contact lenses.

11. The method according to claim 7 where the amniotic material can be either molded or placed in the contact lens material prior to polymerization.

12. The method according to claim 7 where the amniotic material can be either solid, micronized or liquid.
